(19) United States
(12) Patent Application Publication
(10) Pub. No.: US 2004/0074448 A1
(43) Pub. Date: Apr. 22, 2004

(54) Herd control and/or monitoring procedures

(76) Inventors: Craig Robert Bunt, Hamilton (NZ); Peter Stephen Cross, Hamilton (NZ); Rainer Kunnemeyer, Hamilton (NZ); Dale Anthony Carnegie, Hamilton (NZ)

Correspondence Address: JACOBSON HOLMAN PLLC, 400 SEVENTH STREET N.W., SUITE 600, WASHINGTON, DC, 20004, US

(21) Appl. No.: 10/399,863

(22) PCT Filed: Oct. 25, 2001
(86) PCT No.: PCT/NZ01/00236

(30) Foreign Application Priority Data
Oct. 26, 2000 (NZ) 507800

(51) Int. Cl. A01K 1/03

(52) U.S. Cl. 119/421

(57) ABSTRACT

A method of herd management which includes providing each member of the herd with a device (eg; an intra vaginal device) capable of identifying the member, of sensing at least one physiological condition of the member of the herd and communicating with other such devices and/or a base station. With such communication the proximity of devices each other allows groupings to be determined such as a grouping indicative of behavioural oestrus. With such devices it is possible by monitoring groupings of the animal to determine those in oestrus and to have the device administer appropriate active ingredients to some of the animals of the herd.

### TECHNICAL BACKGROUND

The present invention in one aspect recognises a benefit to be derived from the ability to use an intra-vaginal device for animal identification purposes and therefore the present invention is also directed to such a scenario.

The present invention also (in another aspect) relates to methods applicable to a population of members and preferably to herd diagnosis and/or control procedures and/or active agent delivery procedures including related systems and methods.

### BACKGROUND ART

In a population such as that of ruminant mammals there is frequently desire to provide each animal with a device with a view to either eliciting a physiological effect (for example an intra-vaginal device with a view to suppressing oestrus to allow herd oestrus synchrony practises) or to identify/monitor a member of such a population.

The present invention recognises an ability to provide devices powered by appropriate power circuit or circuits which lend themselves to a capability of communicating exteriorly of such an animal carrying the device (preferably using an appropriate antenna and transmitter circuit) which by communication either directly with other devices or some receiver can then convey appropriate information and/or, if desired, be used to allow directly or indirectly some control function to be performed by the device whether instructed by some external receiver or not and whether simply to report a state of affairs to some external receiver or not.

### DISCLOSURE OF THE INVENTION

The present invention also recognises that an intra-vaginal device of any of the passive or active release forms previously disclosed by our company in conjunction with its CIDR™ type passive release devices or those active release devices disclosed in our PCT/NZ98/00011 patent specification can carry within such a device, if desired, circuitry and a battery capable of allowing a measure of communication to or from other such devices or some general communication device such as might interrogate the animals at a gateway or in a yard.

In a first aspect the present invention consists in a method of herd management, which method comprises or includes

providing at least one animal of the herd with a device capable of

sensing a characteristic of the animal,

dispensing an active agent to the animal,

communicating with at least some other device, and

communicating (directly or indirectly, or both) with a monitor externally of the animals of the herd,

having said devices and/or monitor determine by reference to the proximity of the devices to each other and sensed characteristic of the animal which animals are appropriate for the dispensing and/or the halting of the dispensing of an active ingredient, and

dispensing the active ingredient into that or those animals indicated by a said sensed characteristic and/or their grouping.

Preferably said method is such as to determine behavioural oestrus in a herd of animals such as cows.

Preferably said device is in each of the animals of the herd capable of exhibiting a characteristic and dispensing is to a group of which at least one animal has an appropriate characteristic for such dispensing.

In another aspect the present invention consists in an intra-vaginal device having a utility for the animal in question over at least part of a retention period, said device being characterised in that it is provided with transmission and/or interrogatable and/or means externally determinable capable of identifying the recipient animal.

Preferably the device is to be used with an external receiving, interrogator or other animal identification means.

Preferably the identification means includes a transmitter and preferably said transmitter forms part of a communication circuit empowered by a battery having another power draw at least sometime during the retention period therefrom.

Preferably said intra-vaginal device (preferably of a variable geometry retention type) includes an active release feature capable of dispensing at least one pharmacologically effective agent into the vaginal tract of a target mammal into which the device has been inserted.

In still a further aspect the present invention consists in the use within a herd of a plurality of devices in accordance with the present invention, each device including an identifier for a particular animal or a subgroup of such animals of the overall herd or population.

In still a further aspect the present invention consists in apparatus substantially as hereinafter described with reference to the accompanying drawings and related uses and methods.

In one other aspect the invention consists in a method applicable to a population of members which comprises or includes

(i) providing each member of the population with a device in such a way as to identify the member and to sense at lease one physiological condition of the member,

(ii) allowing that device to communicate directly to a receiver (whether a device of another member or via at least one device of another (or other) members(s) or not) to thereby allow some determination for that identified member.

Preferably said determination may occur in the device itself (e.g. if an active agent delivery device as well as some reporting of that determination to the said receiver) or may simply be a diagnosis.

Preferably said device of a member is capable of communicating with at least one other such device of another member of the population and there can be a determination in respect of an identified sub group of the population (for example of a particular physiological condition), for example, if the herd is of mammals capable of exhibiting behavioural oestrus, the devices of a subset of animals within the population that congregate in behaviour indicative of behavioural oestrus can be identified and that can be preferably supported (eg, verified) when at least one sensed physiological condition of a said device of a member is supportive of that condition (e.g. temperature, pressure and/or motion).

Preferably said at least one physiological condition is one or more of temperature, pressure and motion.

In other forms said device is capable of determining directly or indirectly an external condition applicable to the particular member.

Preferably at least some, if not all of said devices each carried by a member, is an intra vaginal device.

Preferably each such intra-vaginal device carries an antenna allowing communication with at least one other device or a receiver for data (whether continuously or as a burst or bursts) from the device.

In a further aspect the present invention consists in, in a herd population, a device retainable in each member of the herd which includes a battery powered circuit or circuits capable of doing at least one or more of the following

(i) transmitting data to some external receiver and/or at least some other device or devices of the herd,

(ii) actively releasing some substance from the device to the animal,

(iii) reporting any such active release of substance to at least one other device of the herd or some external receiver, and

(iv) communicating with at least proximate devices of the herd with a view to render possible a determination of those animals with a device in close proximity that may have and/or be exhibiting an important characteristic (e.g. behavioural oestrus, eating, etc.).

In still a further aspect the present invention consists in any methods of control of herd animals and/or data collection and/or sub grouping of animals in a herd when performed substantially as herein described with reference to any one or more of the accompanying drawings.

In one other aspect the present invention consists in an intra-vaginal device insertable and retainable in the vagina of a target species mammal, said device including an antenna of communication means carried by the device capable of allowing communication to and/or from a communication device externally of the recipient mammal capable of receiving and/or transmitting signals from and/or to said antenna.

Preferably said antenna can at least in part be relied upon for the removal of the device from the vagina of a recipient said target species mammal.

Preferably said device includes a sensor to sense at least one physiological state of a recipient mammal and conveying that sensed condition to a receiver or like device reliant upon the communication means e.g. the communication circuit and said antenna.

Preferably said sensor is capable of detecting one or more of temperature, pressure and motion of a recipient mammal.

Preferably said device is one retainable by its attempting to resume resiliently a condition from which it was deformed in order to allow or facilitate its intra vaginal insertion.

Preferably such retention variable geometry retention is of a kind substantially as herein described with or without reference to the accompanying drawings.

Preferably said intra vaginal device is capable of actively or passively releasing at least one intra vaginally effective agent to the recipient mammal (eg; preferably plunger syringe-like release).

Preferably said device includes at least one active release delivery system for a recipient mammal, such active release being under the action or control of a battery powered circuit, the battery of which is capable of powering the communication circuit of said communication means.

Preferably said antenna is substantially in the form of a flexible tail for the device.

In still a further aspect the present invention consists in the use of an intra-vaginal device in accordance with the present invention.

In still a further aspect the present invention consists in a device of any of the kinds hereinafter described with or without reference to the accompanying drawings and to related methods of use, manufacture and the like.

In still a further aspect the present invention consists in a herd or population management system which relies upon the use of a device in accordance with the present invention intra-vaginally within each animal of the population or herd.

Preferably each said device in conjunction with communications to at least one other device in such a population or herd is capable of indicating for an animal in a sub group of the population or herd a particular behavioural condition, e.g. oestrus.

### DETAILED DESCRIPTION OF THE INVENTION

Preferred forms of the present invention will now be described in conjunction with herd usage (eg; in cows) and with reference to the accompanying drawings in which;

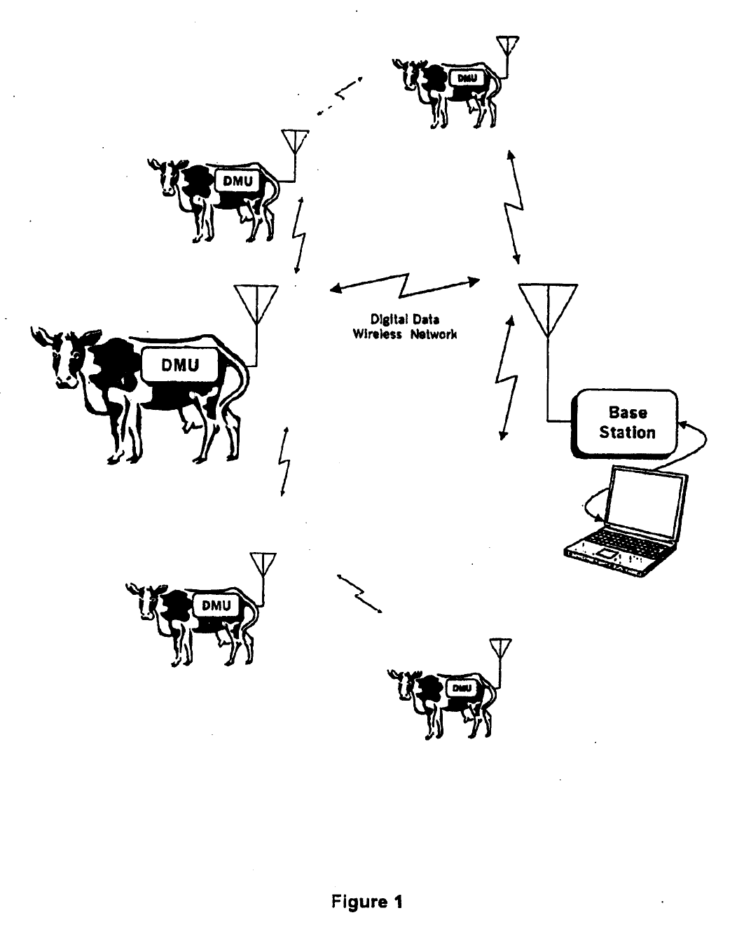

FIG. 1 is a system overview.

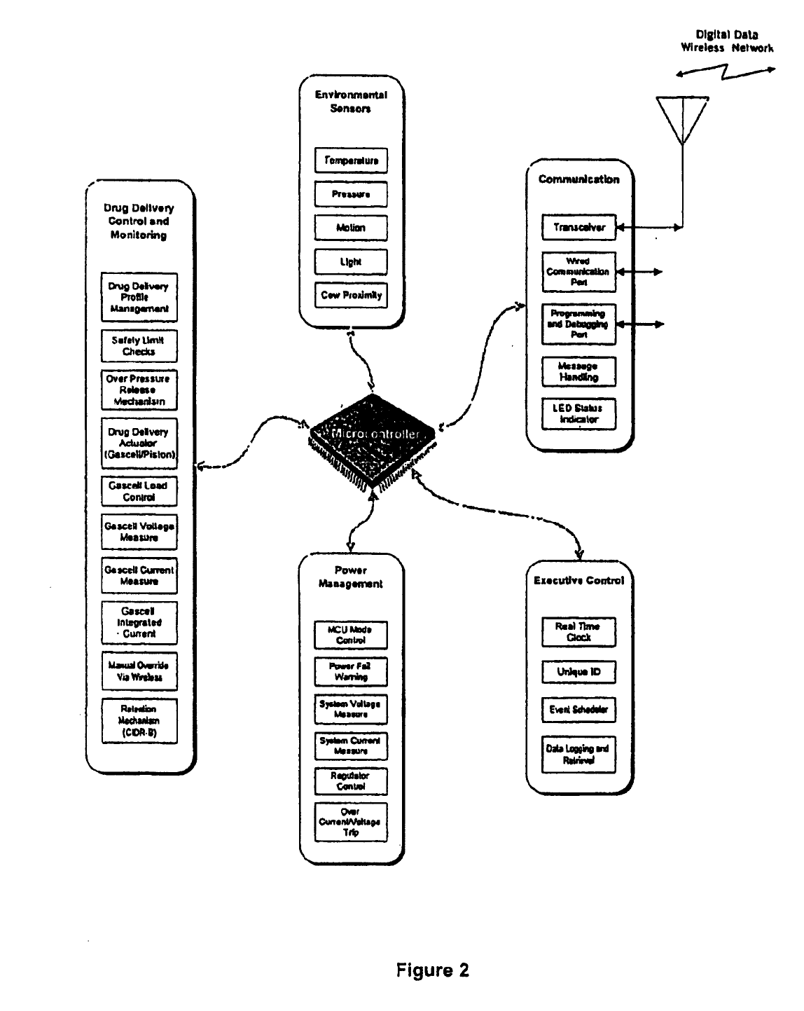

FIG. 2 is a block diagram of delivery and monitor unit (DMU)

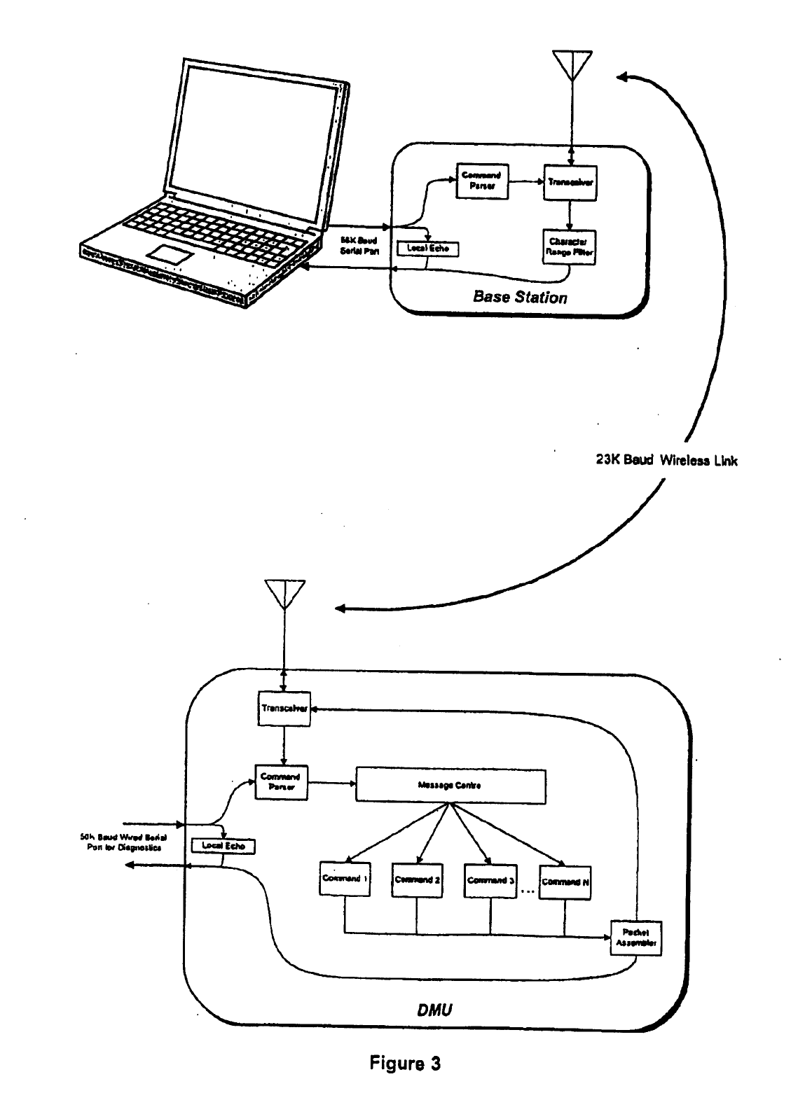

FIG. 3 is a base station/DMU Communication Flow

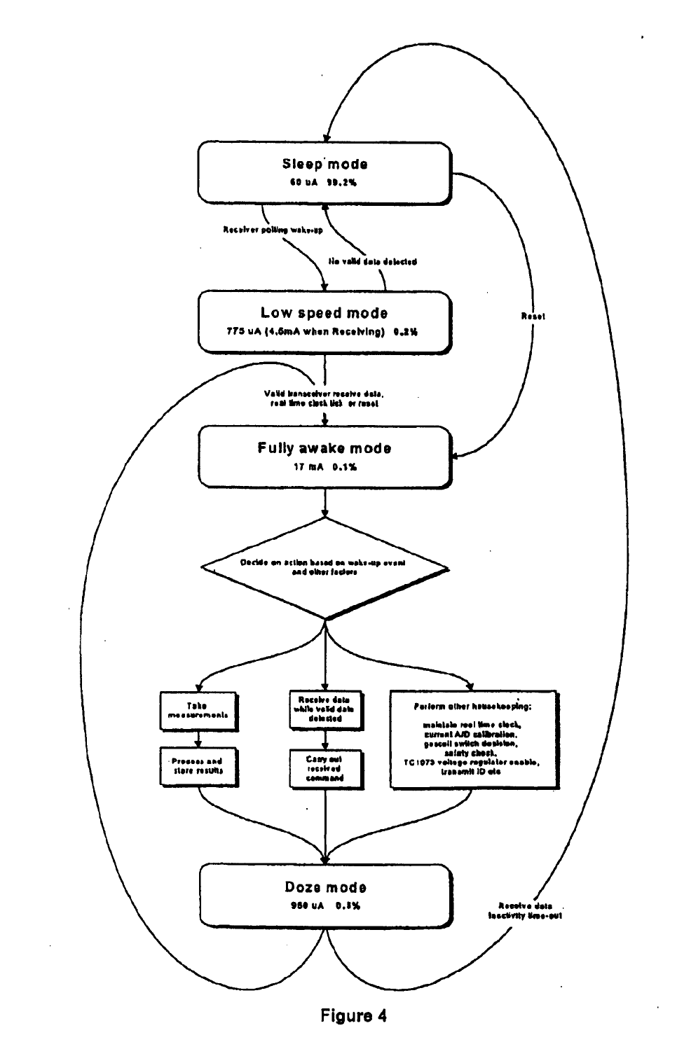

FIG. 4 is a power mode control

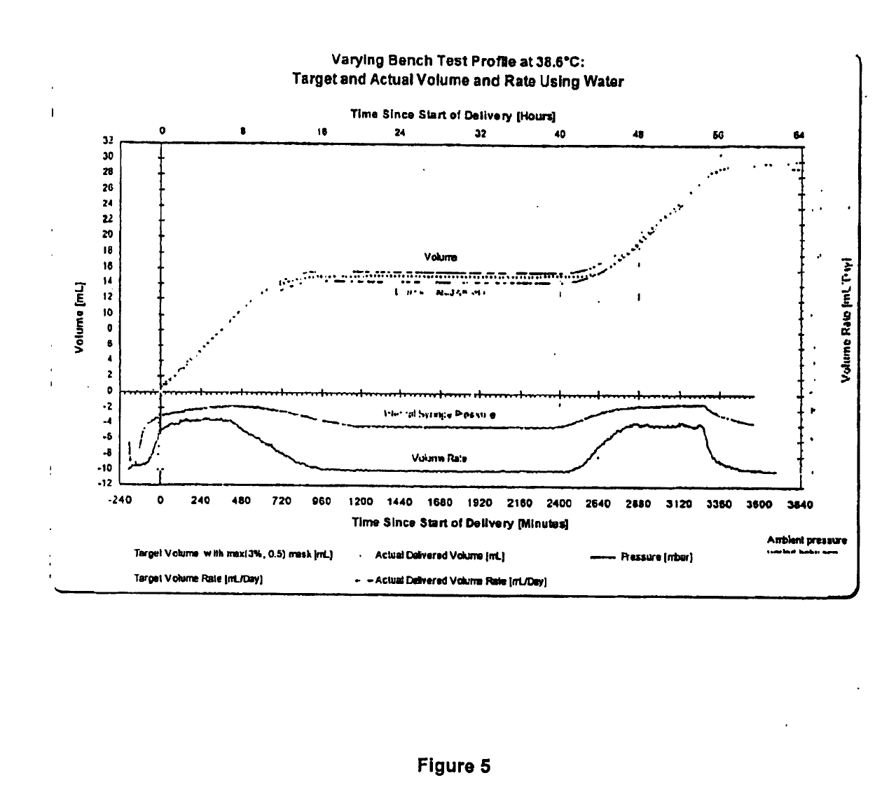

FIG. 5 is a bench test of variable rate delivery

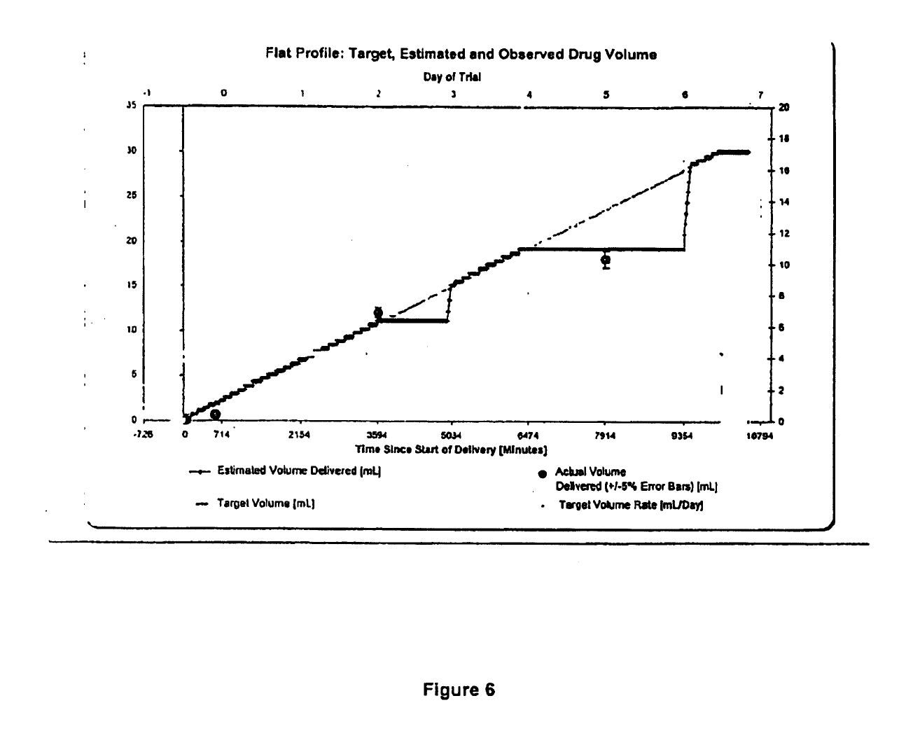

FIG. 6 is a flat profile, target, estimated and observed drug volume

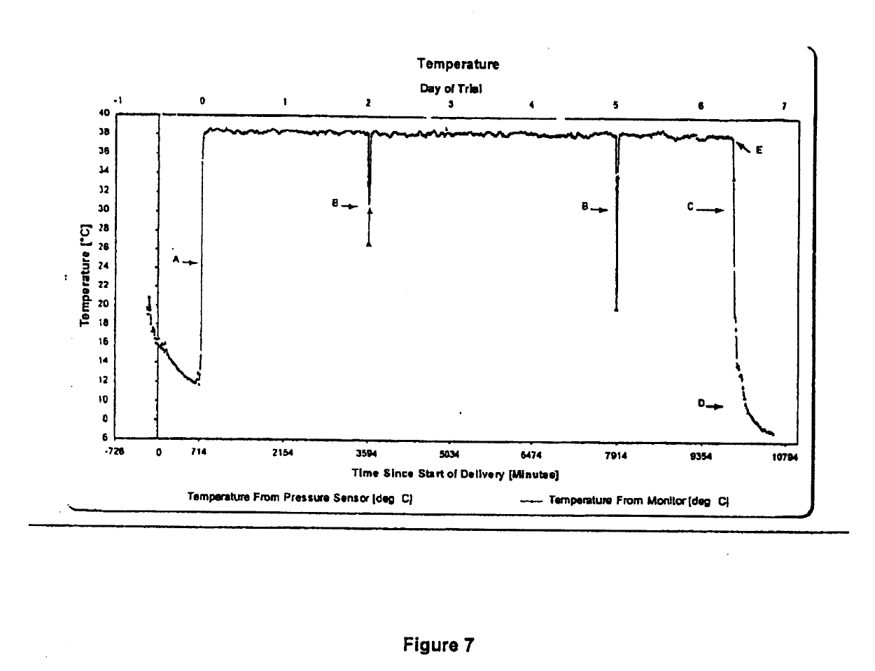

FIG. 7 is a temperature graph

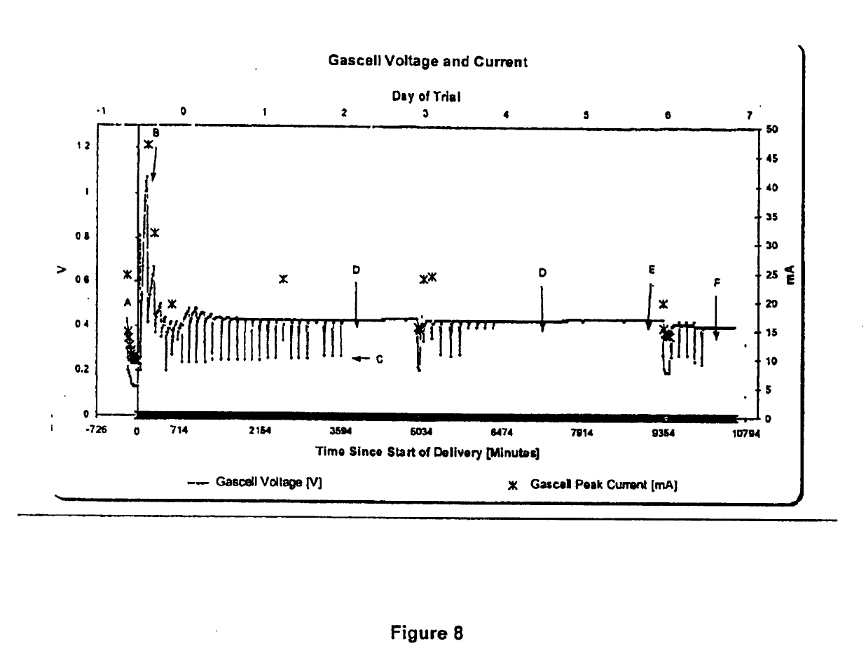

FIG. 8 is a gascell voltage and current graph

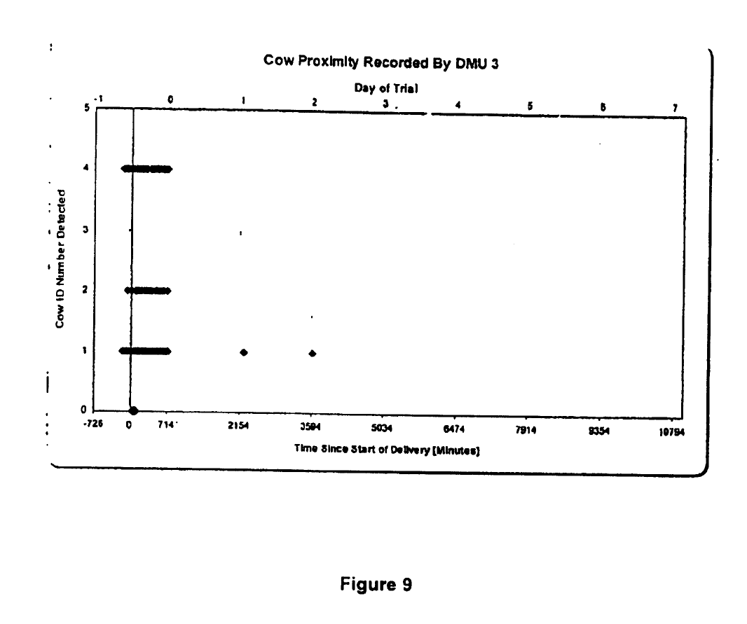

FIG. 9 is a cow proximity recorded by DMU 3

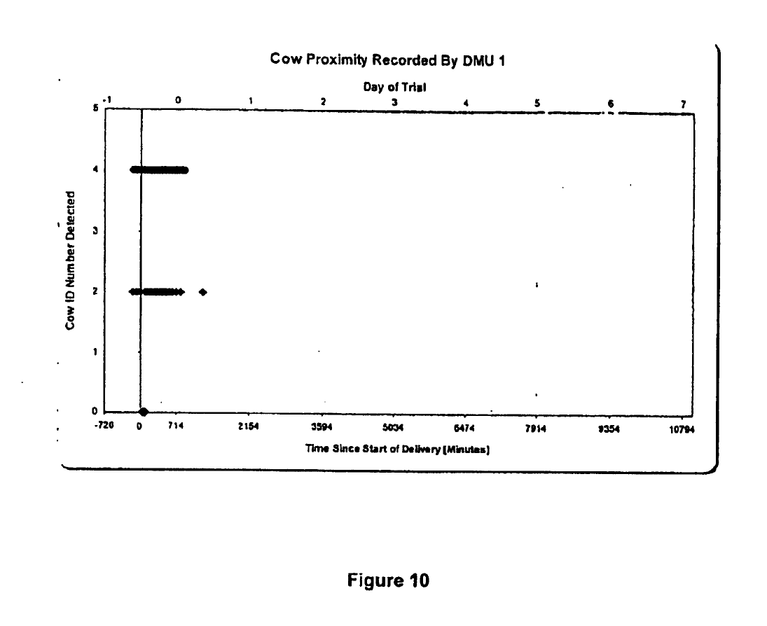

FIG. 10 is a cow proximity recorded by DMU 1

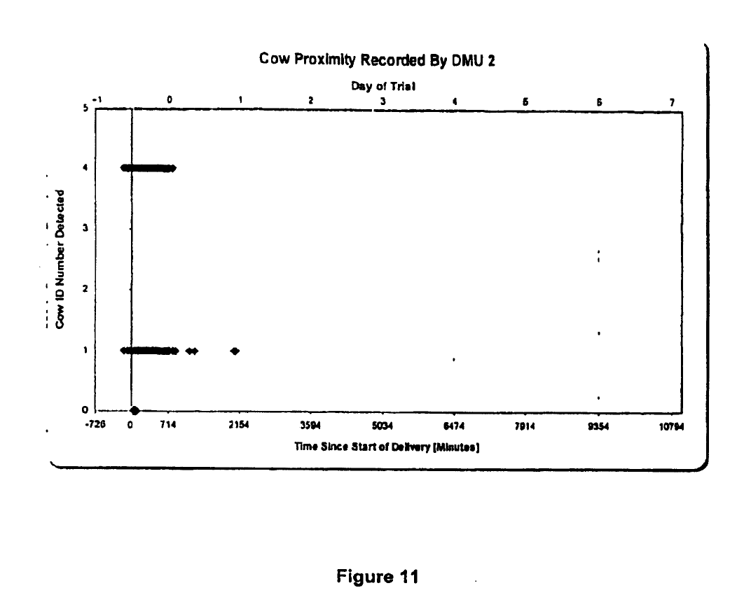

FIG. 11 is a cow proximity recorded by DMU 2

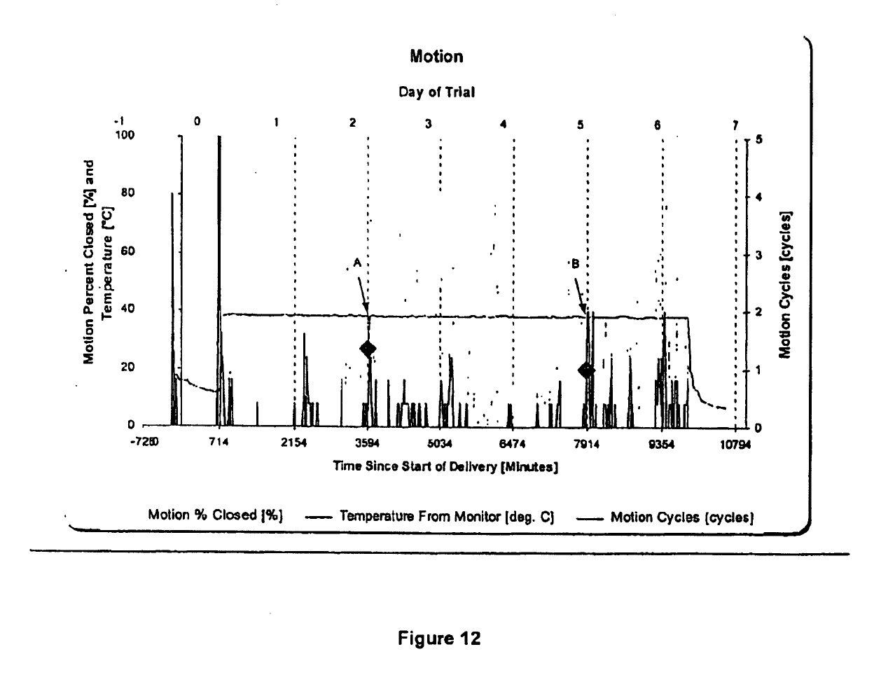

FIG. 12 is a motion graph

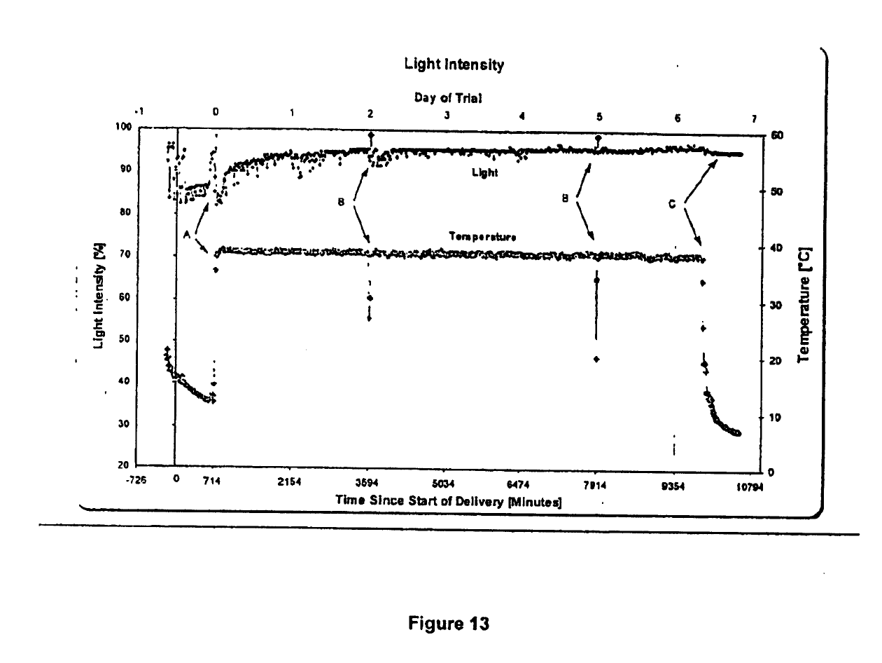

FIG. 13 is a light intensity graph

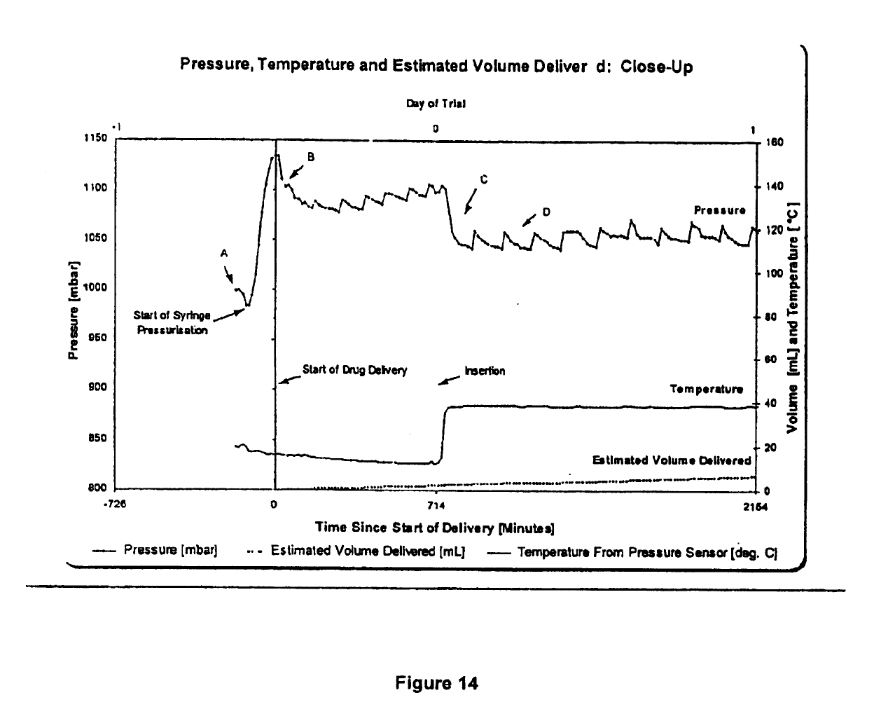

FIG. 14 is a pressure, temperature and estimated volume delivery: closed-up

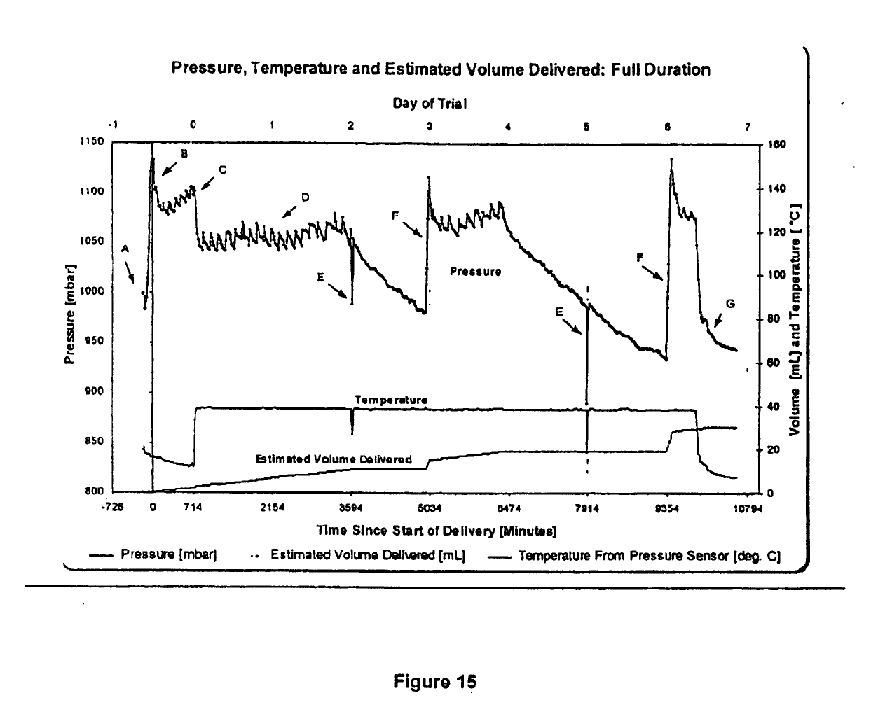

FIG. 15 is a pressure, temperature and estimated volume delivered: full duration

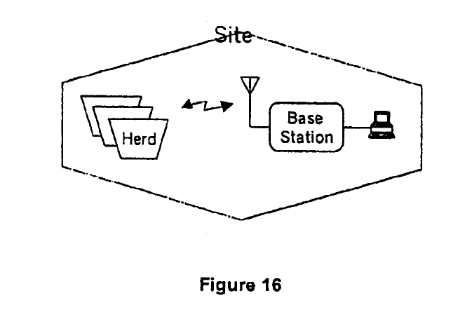

FIG. 16 is a symbolisation of one monitoring site

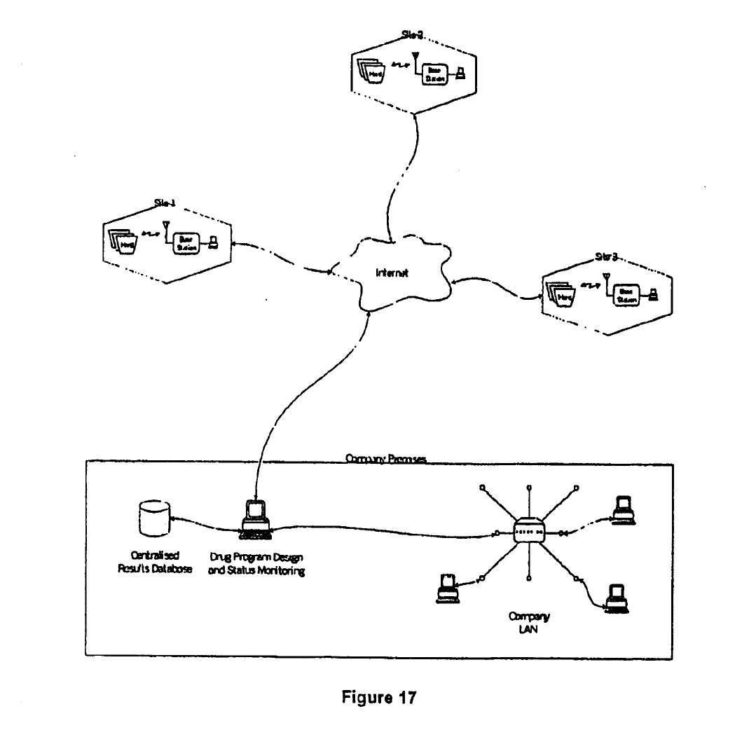

FIG. 17 is a network for remote design and monitoring of drug programs

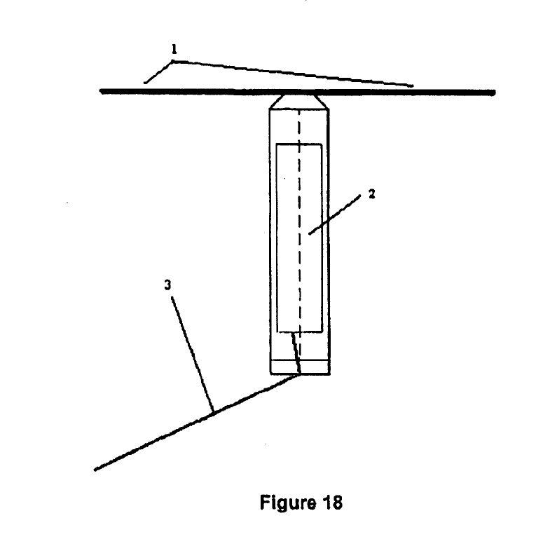

FIG. 18 shows a preferred intra-vaginal device, and

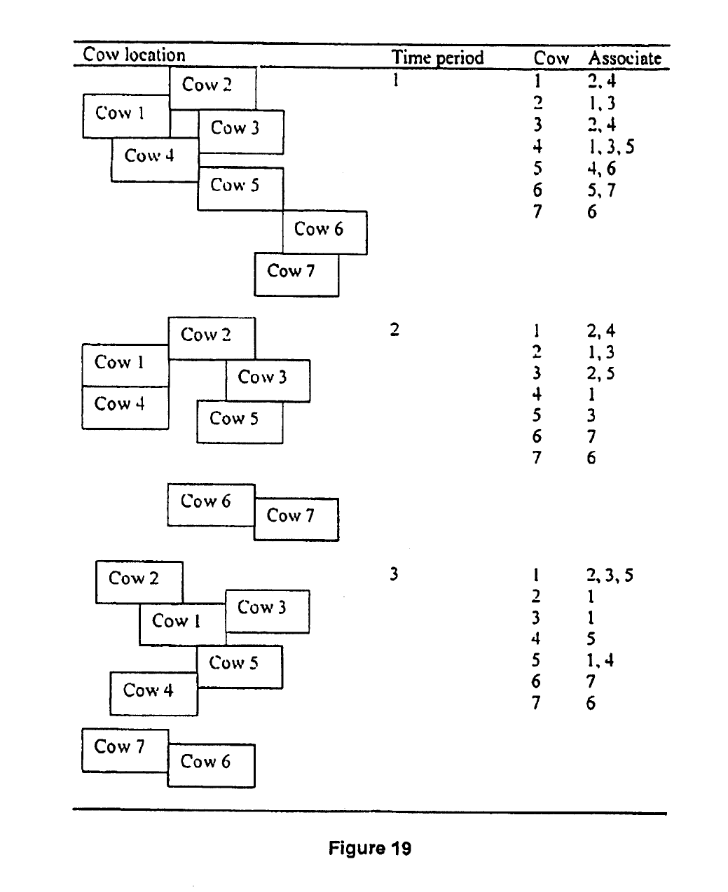

FIG. 19 shows for three time periods various cow groupings and how each cow may be considered as far as association is concerned.

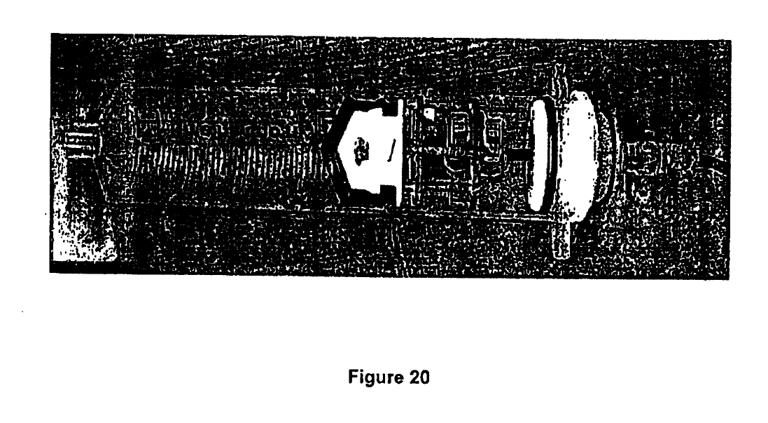

FIG. 20 shows the construction of the DMU component of FIGS. 2 and 18.

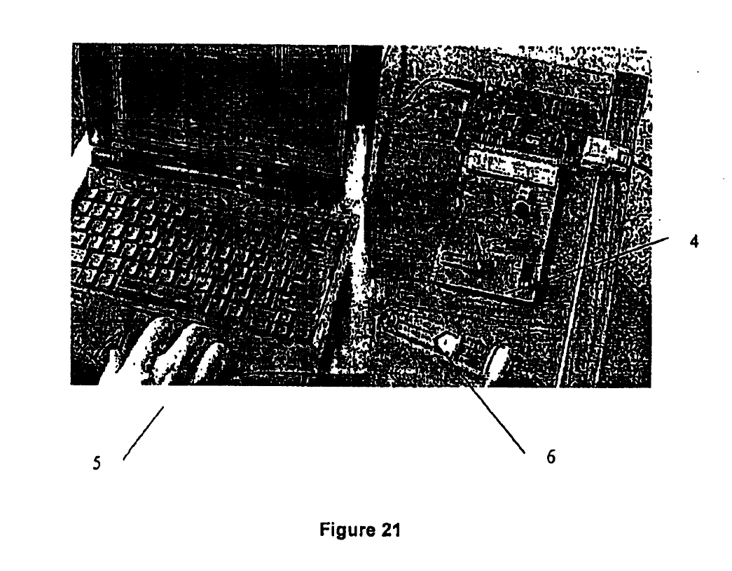

FIG. 21 shows the base station for communication between a DMU and computer.

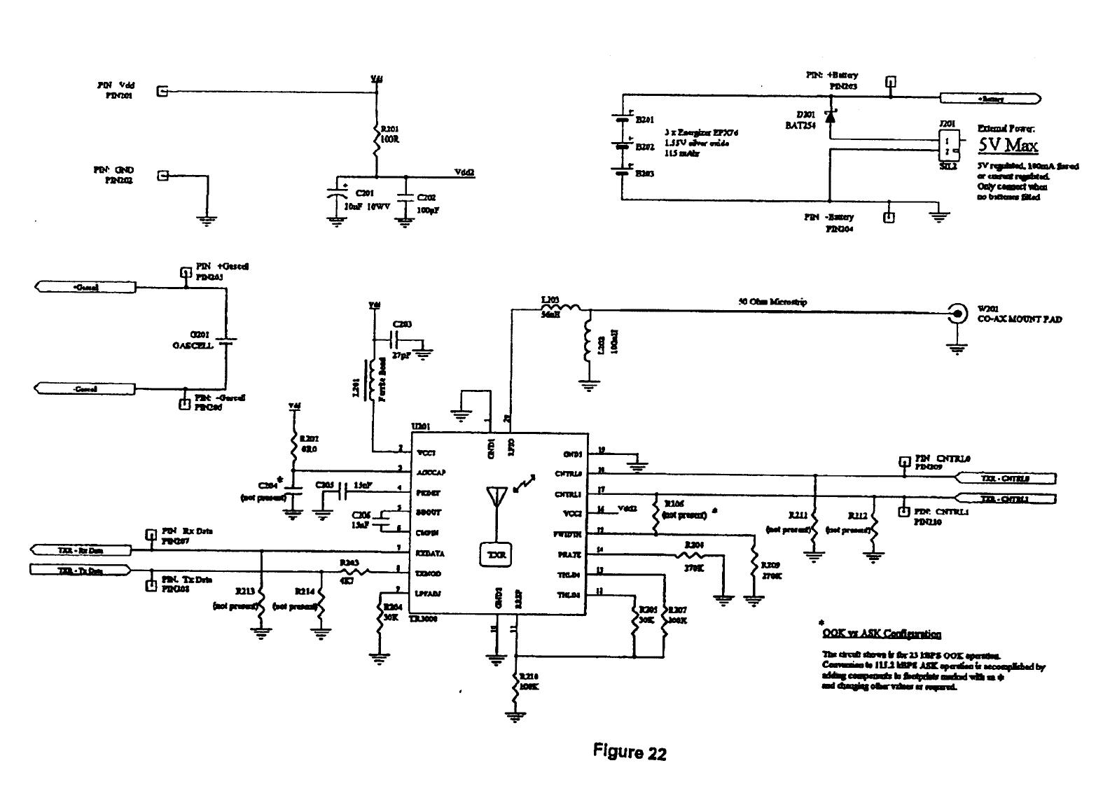

FIG. 22 shows a DMU transceiver and batteries board,

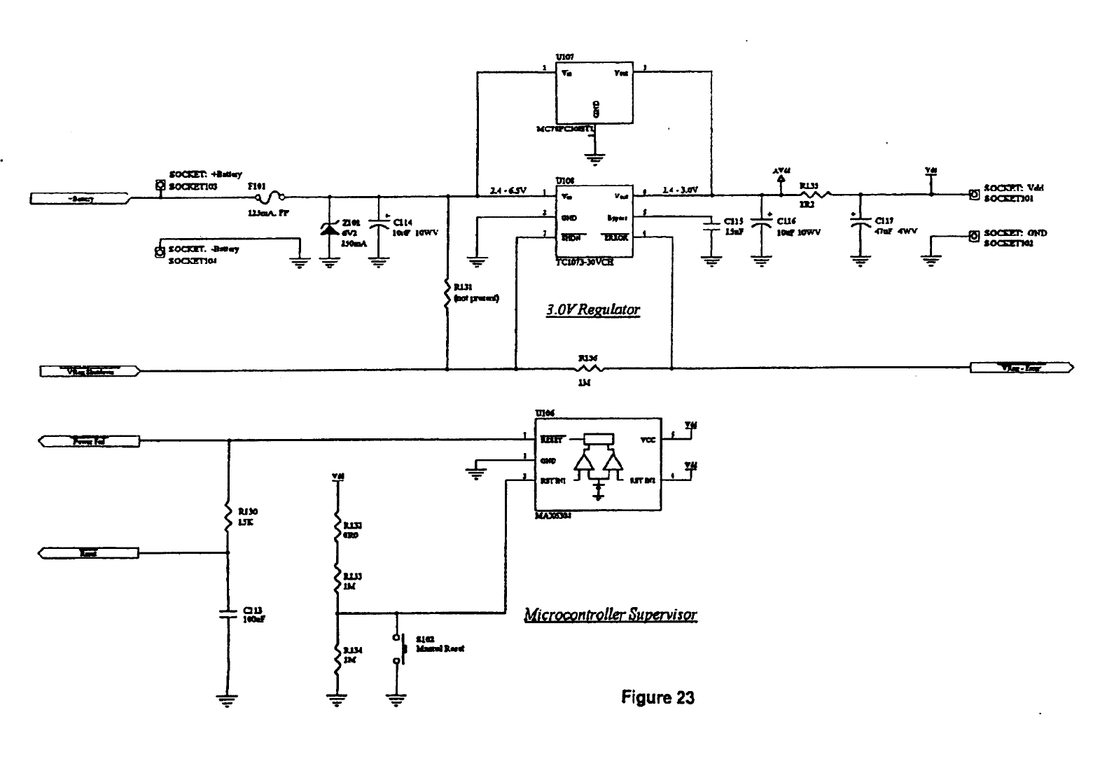

FIG. 23 shows a DMU digital board-power and supervisory,

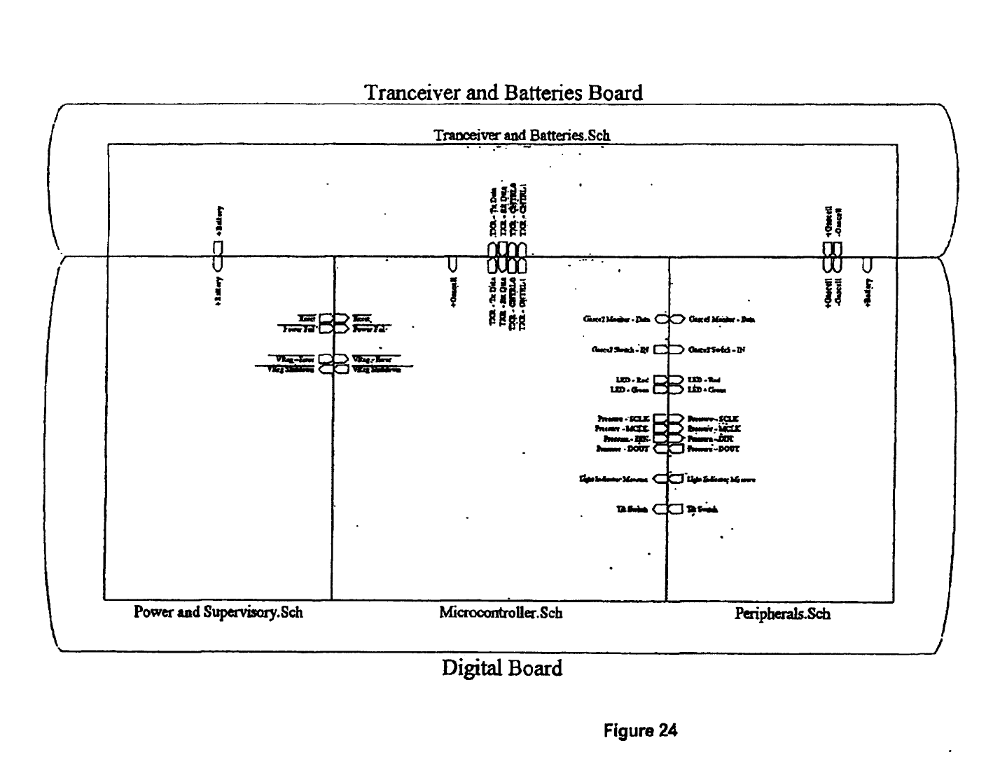

FIG. 24 is the DMU top level showing the relationship between the transceiver and batteries board and the digital board and the separate role of the transceiver and batteries, the power and supervisory, the micro-controller and the peripherals,

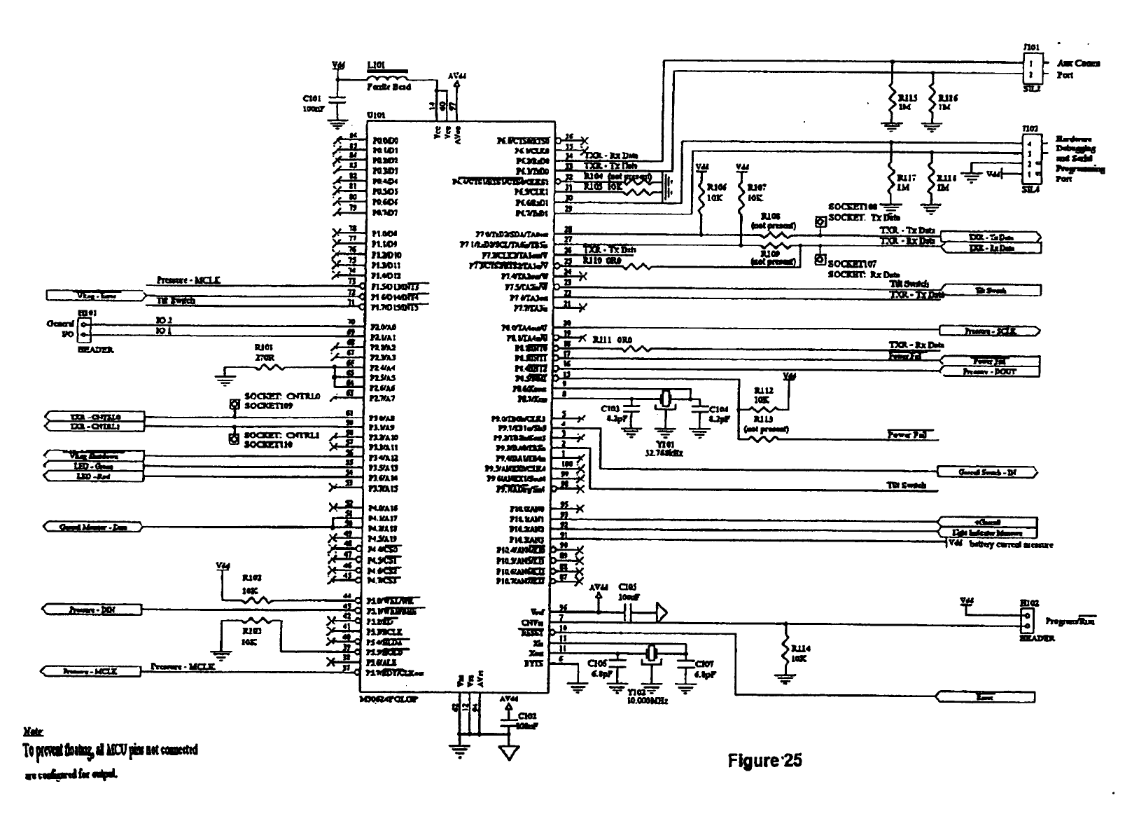

FIG. 25 shows the DMU digital board micro-controller, and

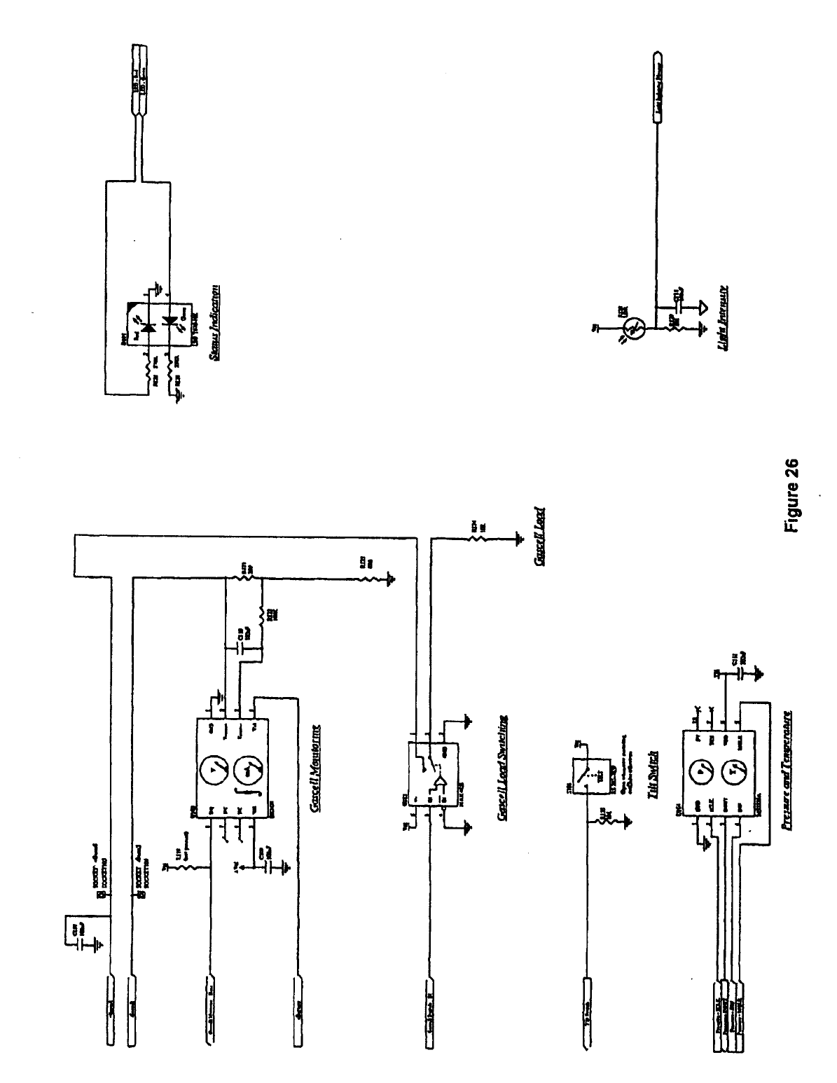

FIG. 26 shows the DMU digital board peripherals, ie; those for gas cell monitoring, gas cell load switching, tilt switch, pressure and temperature and status indication.

Device (FIG. 18) is for insertion into the rumen or vagina of an animal. The device has retention wings (1) attached to a cylindrical body containing electronics for data collection and/or radio communication (2) and an antenna (3).

The base station (4) (FIG. 21) enables communication between a computer (5) and a DMU (6).

The various board and relationship of boards in a DMU are disclosed in FIGS. 22 through 26.

Similar boards have application for the base station with the only difference being that different software would be run on the base station from that on the DMU. Whilst some of the circuitry of a DMU would be redundant in a base station application it nevertheless can provide economics of manufacture and moreover it can allow a base station to be used as a full scale mock up of a DMU for ongoing development work and for ally fault finding.

The system when for herd usage is preferably in the form of a number of drug delivery and control units, each inside a syringe plunger based active agent delivery platform. Preferably dispensing is reliant upon a gas cell (such as that disclosed in WO 94/01165, U.S. Pat. No. 540242 and U.S. Pat. No. 5,741,275) and more preferably when an intra vaginal device such as is in any of U.S. Pat. No. 4,091,807, WO 99/07346, WO 95/13760, WO 98/33452 and WO 96/29025 and/or preferably when using any of the active release arrangements as disclosed in any of our PCT/NZ98/00011, PCT/NZ98/00176, PCT/NZ99/00083 and PCT/NZ00/00155.

On-board sensors monitor the condition of the animal and the electronics package. Another sub-system provides a short range, wireless half-duplex digital data link. The radio frequency carrier of 433.82 MHz is modulated with on-off keying at a symbol rate of 19.2KBaud. The range is approximately 60 m outdoors, line of sight.

### INTER DEVICE RANGE ESTIMATION

Output power is constant over the life of the battery. However, for a fixed distance, some variation of received signal strength is to be expected due to variations in build, component tolerances, placement in the animal and particular propagation effects (such as multipath fading). There is no direct indication of received signal strength from the transceiver integrated circuit, but received signal strength can be estimated using one of the following techniques:

Over-sample the data bits being received. Once the receiver clock has synchronised with the transmitter clock, each sample should be of the same value throughout the duration of the bit period. As received signal strength decreases, the ratio of sample values will approach 0.5.

Transmit at different powers with a description of the output power level contained as part of the payload in the data packet.

The use of one of these techniques allows an estimation of distance to be obtained. The more transmissions used over a short period, the more accurate the estimate.

### ADVANTAGES OF DUPLEX COMMUNICATION

The ability of duplex communication between animals has various advantages over simplex communication.

Commands can be sent to the in-animal device with a confirmation reply being returned, indicating that the command was received and understood. This greatly improves reliability.

If necessary, a high degree of security can be provided using standard interrogative encryption algorithms.

Inter-animal communication is possible (discussed further below).

Facilitates efficient use of the communication channel. Slotted communication in time division multiple access schemes have less blocking under heavy load and can be configured for guaranteed bandwidth, which is not the case with random access schemes. E.g. Token Ring vs. Ethernet local area network topology. Slotted communication schemes require two-way communication.

### USES OF THE NETWORK ONLY POSSIBLE WITH DUPLEX COMMUNICATION

### Inter-Animal Algorithms in Real Time

The information transmitted between animals could immediately alter the behaviour of the devices present in other animals. For example, if a robust drug delivery technique was available to slow or accelerate the oestrus cycle, individual cows could be retarded or advanced toward the average known cycle state in a real time process. Oestrus would then occur at the same time for all cows in the herd.

### Establishing Grouping Behaviour

Current behavioural recording systems such as tail-paint, MountCount and HeatWatch rely on the physical interaction between cows in heat, i.e. contact during mounting behaviour. They miss loner cows that prefer not to have social interaction during this time. With a duplex system, it is possible to record the grouping of animals throughout the day. This will detect all types of grouping behaviour including loner cows.

More subtle behavioural patterns can be established by combining the data from the on-board sensors with the grouping data. For example, if a cow does not have an increase in vaginal temperature, but is socialising with the group of cows which are in heat, and participating in mounting behaviour, this would allow a study to target of cows of that nature.

The absolute position of the animals cannot be worked out from the data set, nor is any individual data element a reliable indication of the range between animals involved in a data exchange. However, by analysing enough exchanges over time, an accurate statistical representation of animal grouping will be discerned over a time period of several minutes (see FIG. 22).

### TRANSMISSION PROTOCOLS TO ESTABLISH GROUPING BEHAVIOUR

The grouping information can be accomplished with a number of different techniques, three of which are now described:

1. Broadcast One-Hop Passive: At regular intervals, each animal asynchronously transmits its ID number to all those around it using a broadcast packet. Those receptions are logged with the payload which contains the time and ID number of the sender. Received signal strength is also logged for each packet. When the animals are next in range of the base station or portable data gathering unit, a central database is updated which provides a master record of all transmissions. The base station has graphing capabilities to visualise which animals were grouped together.

2. Polling One-Hop Interrogation: As for Broadcast One-Hop Passive, but transmissions are addressed individually to each animal which has not been heard from recently (within the last 20 minutes for example). Receivers reply to the sender with their ID number. If an interrogation reply is received, they are not interrogated again for another 20 minutes. The sender notes the replies that it gets in the standard manner. The receiver logs the results of all interrogations. Although more complex, this modification more accurately determines received signal strength (since there are two transmissions per exchange along the same path) and reduces the amount of network traffic (since close animals are not unnecessarily addressed).

3. Broadcast Multi-Hop Hot Potato: At regular intervals, each animal asynchronously transmits its ID umber to those around it. Those receptions are logged with the payload containing the received signal strength, time the original packet was transmitted and ID number of the originating animal. The ID number of the receiving animal is appended to the data, and it is rebroadcast after a time delay based on the ID number of the receiving animal (so that collisions do not occur with other animals responding in the same way). If an animal receives a packet and it sees it's own ID in the payload, it does nothing. This prevents data packets from continually circulating throughout the herd. This data is of the same quality and can be processed in a similar manner as with the one-hop passive technique. The originator also logs any of the forwarded packets that originated from itself for another estimation of animal grouping. The packet may take one of several paths, or even arrive via multiple paths to a given animal. Only the first packet to arrive is processed if there is a command present, but the signal strengths of any subsequent packets are logged.

The rate of transmission is reduced during the night to conserve battery power.

### DEFINITION OF TERMS

Broadcast: A transmission from one sender intended for multiple receivers. All receivers process this type of packet in which the address field set to a value indicating that the packet is a broadcast packet. Also referred to as multicast.

Polled: A transmission from one sender intended for one receiver. Other receivers may hear the transmission but will not process this type of packet unless the address field is set to a value indicating that the packet is for them.

One-hop: The channel consists of one direct transmission from a single sender to one receiver.

Multi-hop: The channel consists of a number physical links. The packet is stored and forwarded to the next link by repeaters. This may happen one or times until it reaches the destination. The packet may take one of several paths, or even arrive via multiple paths. Only the first packet to arrive is processed.

Simplex: Communication in one direction only.

Full-Duplex: Communication in both directions simultaneously.

Half-Duplex: Communication in both directions, but not simultaneously.

Duplex: full-duplex or half-duplex

FIG. 1 shows the use of the delivery and motor unit (DMU) of the present invention in conjunction with a grouping of cows showing the system with its relationship between DMUs and a base station. FIG. 2 shows how the DMU committed as far as logic is concerned to its micro controller operates. FIG. 2 being a block diagram of the DMU shows a capability in respect of the sectors of power management, executive control, communication, environmental senses and drug delivery control and monitoring.

As can be seen front FIG. 2 each of the sectors of function assumed by the micro controller can be further segmented down into the areas depicted.

FIG. 3 shows how a base station might communicate with an individual DMU using, for example, a 56K Baud Serial Port and a 23K Baud Wireless Link to the DMU.

FIG. 4 shows power mode control with reference to a sleek mode, a low speed mode, a fully awake mode and a doze mode and the activations resets and decision making involving transition from one to another.

FIG. 5 shows a bench test of variable rate delivery in respect of (by way of example) the expression of water from a intra vaginal device.

FIG. 6 shows the target estimated and observed drug volume over a 7-day period for a device inserted intra vaginally

FIG. 7 shows also against time since the start of delivery the temperature over the time period, its temperature being that of the vagina during a 7-day period for a device insert intra vaginally.

FIG. 8 shows gascell voltage and current against time since the start of delivery.

FIG. 9 shows cow proximity recorded by DMU 3.

Many successful transmissions prior to insertion were recorded. After that time, only two inter-cow transmissions were seen by DMU 3. With the transceiver on DMU 2 only having a limited range, DMU 4 not responding after insertion and DMU 2 unable to respond after day 2, only inter-cow transmissions from DMU 1 were recorded by DMU 3. Since DMU 1stopped responding after day 2, there were only.

In these cow proximity graphs, the base station is represented with an ID number of zero. The reception just after time equals zero is a manual test initiated by the base station.

Obviously, a DMU cannot receive a transmission from itself when it is busy transmitting, which is why there are no transmission recorded from DMU 3.

FIG. 10 is cow proximity recorded by DMU 1.

FIG. 11 is cow proximity recorded by CUM 2.

FIG. 12 shows motion against time since the start of delivery. All motion stopped when the DMU fell out. The fact that motion cycle peaks coincide predominately with high vales of. To make proper sense of all, a proper observation record needs to be kept. Motion cycles has an arbitrary scaling and will be increased to provide better resolution

FIG. 13 shows light intensity against time since the start of delivery.

It was unfortunate that the LED was programmed to turn on while light readings were taken. Even so, the light intensity readings are of some value and indicate that a simple light sensor can be used to assist in a self-diagnosis role for the drug delivery hardware.

It was a surprise to see that light varied so much in-vivo. It is suggested that this is caused by the LED providing total internal reflection from the syringe wall. If confirmed, this might lead to a simple method of detecting what kind of material is in contact with the syringe near the LED, be it gas, flesh, or a thick layer of clear or opaque mucus.

The grid lines are equally spaced and are approximately aligned with the observation times. Observation time was at 0815±45 minutes.

The solid black vertical axis at time equals zero on all the graphs represents the beginning of drug delivery. Negative time before that point represents the period after power on reset during which the syringe pressurises. After time equals zero, the delivery programme commences. The vehicle was delivered for 11 hours

FIG. 14 shows pressure, temperature and estimated volume delivered.

FIG. 15 shows pressure, temperature and estimated volume delivered for the full duration.

FIG. 16 shows symbolism that might be used in conjunction with the teaching of the invention for one monitoring site.

FIG. 17 shows a network for remote design and monitoring of a drug program showing sites 1, 2 and 3 (each of the kind shown in FIG. 16) and showing there below but linked between the various sites on the internet a company premise.

FIG. 20 shows the construction of a preferred device as shown in FIG. 18.

FIG. 21 shows the base station for communication between the device as shown in FIG. 20 and a computer.

1. A method of herd management, which method comprises or includes
providing at least one animal of the herd with a device capable of
sensing a characteristic of the animal,
dispensing an active agent to the animal,
communicating with at least some other device, and
communicating (directly or indirectly, or both) with a monitor externally of the animals of the herd,
having said devices and/or monitor determine by reference to the proximity of the devices to each other and sensed characteristic of the animal which animals are appropriate for the dispensing and/or the halting of the dispensing of an active ingredient, and
dispensing the active ingredient into that or those animals indicated by said sensed characteristic and/or their grouping.

2. A method of claim 1 wherein said method is such as to determine behavioural oestrus in a herd of animals such as cows.

3. A method of claim 1 or 2 wherein said device is in each of the animals of the herd capable of exhibiting a characteristic and dispensing is to a group of which at least one animal has an appropriate characteristic for such dispensing.

4. An intra-vaginal device having a utility for the animal in question over at least part of a retention period, said device being characterised in that it is provided with transmission and/or interrogatable and/or means externally determinable capable of identifying the recipient animal.

5. A device of claim 4 to be used with an external receiving, interrogator or other animal identification means.

6. A device of claim 4 or 5 wherein the identification means includes a transmitter and preferably said transmitter forms part of a communication circuit empowered by a battery having another power draw at least sometime during the retention period therefrom.

7. A device of any one of claims 4 to 6 which is of a variable geometry retention.

8. A device of any one of claims 4 to 7 which includes an active release feature capable of dispensing at least one pharmacologically effective agent into the vaginal tract of a target mammal into which the device has been inserted.

9. A device of any one of claims 4 to 8 substantially as herein described with reference to FIGS. 18 and 20.

10. The use within a herd of a plurality of devices in accordance with any one of claims 4 to 9, 20, 22 to 31 and 34, each device including an identifier for a particular animal or a subgroup of such animals of the overall herd or population.

11. A method applicable to a population of members which comprises or includes
(i) providing each member of the population with a device in such a way as to identify the member and to sense at lease one physiological condition of the member,
(ii) allowing that device to communicate directly to a receiver (whether a device of another member or via at least one device of another (or other) members(s) or not) to thereby allow some determination for that identified member.

12. A method of claim 11 wherein said determination may occur in the device itself (e.g. if an active agent delivery device as well as some reporting of that determination to the said receiver) or may simply be a diagnosis.

13. A method of claim 11 or 12 wherein said device of a member is capable of communicating with at least one other such device of another member of the population and there can be a determination in respect of an identified sub group of the population.

14. A method as claimed in any one of claims 11 to 13 wherein said at least one physiological condition is of a herd of mammals capable of exhibiting behavioural oestrus.

15. A method of claim 14 wherein a subset of animals within the herd that congregate in behaviour indicative of behavioural oestrus are identified and that identification is verifiable by at least one said device sensed physiological condition.

16. A method as claimed in claim 14 wherein said at least one physiological condition is one or more of temperature, pressure and motion.

17. A method as claimed in claim 14 wherein such a device is capable of determining directly or in directly an external condition applicable to the particular member.

18. A method as claimed in any one of claims 11 to 17 wherein at least some, if not all, of said devices each carried by a member, is an intra vaginal device.

19. A method as claimed in claim 18 wherein each such intra-vaginal device carries an antenna allowing communication with at least one other device or a receiver for data (whether continuously or as a burst or bursts) from the device.

20. In a herd population, a device retainable in each member of the herd which includes a battery powered circuit or circuits capable of doing at least one or more of the following
(i) transmitting data to some external receiver and/or at least some other device or devices of the herd,
(ii) actively releasing some substance from the device to the animal,
(iii) reporting any such active release of substance to at least one other device of the herd or some external receiver, and
(iv) communicating with at least proximate devices of the herd with a view to render possible a determination of those animals with a device in close proximity that may have and/or be exhibiting an important characteristic (eg; behavioural oestrus, eating, etc.).

21. Methods of control of herd animals and/or data collection and/or sub grouping of animals in a herd when performed substantially as herein described with reference to any one or more of the accompanying drawings.

22. An intra-vaginal device insertable and retainable in the vagina of a target species mammal, said device including an antenna of communication means carried by the device capable of allowing communication to and/or from a communication device externally of the recipient mammal capable of receiving and/or transmitting signals from and/or to said antenna.

23. A device as claimed in 22 wherein said antenna can at least in part be relied upon for the removal of the device from the vagina of a recipient said target species mammal.

24. A device as claimed in 22 wherein said device includes a sensor to sense at least one physiological state of a recipient mammal and conveying that sensed condition to a receiver or like device reliant upon the communication means e.g. the communication circuit and said antenna.

25. A device as claimed in 22 wherein said sensor is capable of detecting one or more of temperature, pressure and motion of a recipient mammal.

26. A device as claimed in 22 wherein said device is one retainable by its attempting to resume resiliently a condition from which it was deformed in order to allow or facilitate its intra vaginal insertion.

27. A device as claimed in 22 wherein such retention variable geometry retention is of a kind substantially as herein described with or without reference to the accompanying drawings.

28. A device as claimed in any one of claims 22 to 27 wherein said intra vaginal device is capable of actively or passively releasing at least one intra vaginally effective agent to the recipient mammal.

29. A device as claimed in 28wherein said device has a plunger syringe-like release.

30. A device as claimed in 22 wherein said device includes at least one active release delivery system for a recipient mammal, such active release being under the action or control of a battery powered circuit, the battery of which is capable of powering the communication circuit of said communication means.

31. A device as claimed in 22 wherein said antenna is substantially in the form of a flexible tail for the device.

32. Apparatus substantially as hereinafter described with reference to the accompanying drawings and related uses and methods.

33. The use of an intra-vaginal device of any one of claims 20, 22 to 31 and 34.

34. A device of any of the kinds hereinafter described with or without reference to the accompanying drawings and to related methods of use, manufacture and the like.

35. A herd or population management system which relies upon the use of a device in accordance with claim 33 intra-vaginally within each animal of the population or herd.

36. A system as claimed in claim 35 wherein each said device in conjunction with communications to at least one other device in such a population or herd is capable of indicating for an animal in a sub group of the population or herd a particular behavioural condition, e.g. oestrus.
